(19) United States
(12) Patent Application Publication
(10) Pub. No.: US 2026/0062758 A1
(43) Pub. Date: Mar. 5, 2026

(54) GENDER-NEUTRAL URINE-BASED MIRNA TEST FOR DETECTING UROLOGICAL CANCERS

(71) Applicant: NOTHING CREATIVE LLC, White Sulphur Springs, WV (US)

(72) Inventors: Brian Joseph Hajjar, White Sulpher Springs, WV (US); Ummar Raheel, Wayne, NJ (US)

(21) Appl. No.: 19/314,369

(22) Filed: Aug. 29, 2025

Related U.S. Application Data
(60) Provisional application No. 63/689,247, filed on Aug. 30, 2024.

(51) Int. Cl. C12Q 1/6886

(52) U.S. Cl. CPC C12Q 1/6886; C12Q 2600/158; C12Q 2600/178; G01N 2800/50

(57) ABSTRACT

The present invention provides a gender-neutral, non-invasive diagnostic method for detecting urological cancers, including prostate, bladder, kidney, renal pelvis, and testis cancers, using urine-based microRNA (miRNA) biomarkers. The method comprises obtaining a self-collected urine sample, extracting miRNA, and quantifying biomarker expression levels using, e.g., reverse transcription quantitative PCR (RT-qPCR), next-generation sequencing (NGS), or microarray analysis. A panel of miRNAs, e.g., hsa-let-7b-5p, hsa-miR-26b-5p, hsa-miR-145-5p, hsa-miR-4253p, hsa-miR-195-5p, hsa-miR-203a-3p, hsa-miR-30c-5p, and hsa-miR-30a-3p, enables sensitive and specific discrimination between cancer and non-cancer patients. The invention is capable of high diagnostic performance and accuracy. The assay may be highly reproducible. This present invention provides a cost-effective, non-invasive, inclusive platform applicable across cisgender, transgender, and non-binary populations, facilitating early cancer detection and improved healthcare accessibility.

### CROSS REFERENCE TO RELATED APPLICATIONS

This application claims the benefit of U.S. Provisional Application No. 63/689,247, filed on Aug. 30, 2024, the entire disclosure of each of which is incorporated by reference herein.

### FIELD OF THE INVENTION

The present invention relates to a method and system for detecting urological cancers including that of prostate, uterine, bladder, testis, and kidney using urine-based microRNA (miRNA) analysis. More specifically, the invention pertains to a non-invasive diagnostic test capable of identifying individuals of all genders with urological cancers or those with high risk of developing a urological cancer by analyzing specific miRNA biomarkers present in their self-collected first-void urine samples.

### BACKGROUND OF THE INVENTION

Urological cancer is any cancer that starts in the urinary or reproductive tract organs. The most common sites of urological cancer are prostate, bladder, kidney, and testis.

According to GLOBOCAN 2022 prostate, bladder, kidney and testis cancers have ranked 2^{nd}, 6^{th}, 8^{th}, and 23rd respectively in United States. Between 2013 and 2017, urologic cancers accounted for one-third of all cancers diagnosed in men, according to the CDC. Out of the 302,304 cases diagnosed annually, 67% were prostate cancer, 19% were urinary bladder cancer, 13% were kidney or renal pelvis cancer, and 3% were testicular cancer.

Prostate cancer stands as the most prevalent form of cancer among cisgender men, aside from skin cancer. Its incidence exceeded 1.4 million cases in 2020, leading to 375,000 deaths globally.

The understanding of urological cancers in individuals with other genders such as transgender females who are assigned male at birth, remains limited. Current guidelines are gender specific, leaving gaps in transgender and non-binary populations who may still be at risk (e.g. prostate cancer in transgender women assigned male at birth). Unlike the comprehensive guidelines available for screening, diagnosis, management, and outcomes in cisgender males, clinicians lack evidence-based guidance for managing urological cancers including prostate cancer in transgender women.

Early detection of urological cancers is crucial for effective treatment and improved patient outcomes. Current diagnostic methods for detecting urological cancers, such as prostate-specific antigen (PSA) testing and digital rectal examination (DRE) for diagnosing prostate cancer, suffer from limitations such as low specificity and invasiveness. Furthermore, many guidelines for screening urological cancers are tailored to specific genders, often excluding transgender women and other non-binary individuals.

There is a clear unmet need for: a gender-neutral, inclusive test that provides a self-sampling, non-invasive platform that patients can utilize at home, or in a location of their choosing, designed with molecular biomarkers with high specificity and sensitivity across patient populations

This highlights the need for developing a non-invasive, highly sensitive, and specific diagnostic tests for screening urological cancers including prostate cancer in individuals of all genders. This is crucial for ensuring comprehensive healthcare and inclusivity in cancer screening practices.

### SUMMARY OF THE INVENTION

The present invention discloses a miRNA-based gender-neutral test for detecting urological cancers in self-collected urine samples from male, female and non-binary patients. The test involves the analysis of specific miRNA biomarkers levels present in urine samples collected from individuals suspected of having urological cancer or having a high risk of developing any urological cancer.

### BRIEF DESCRIPTION OF THE DRAWINGS

FIG. 1 is a schematic of an exemplary embodiment of the miRNA-based gender-neutral test for detecting urological cancers in accordance with the invention.

### DETAILED DESCRIPTION OF THE INVENTION

As used herein the term “Transgender” refers to a person whose gender identity differs from their assigned gender at birth. The term “non-binary” is meant to include any person whose gender identity does not fit the traditional categorical definitions of gender. This term encompasses gender identities in which a person may identify as both male and female, neither and all spectrums of gender identity in between.

In accordance with the invention, urine may be obtained from any individual. The individual may be healthy and without any known disease. Alternatively, an individual may be a person suspected of having a disease. Preferably, urine samples are collected in sterile containers to minimize the possibility of contamination by environmental microorganisms or foreign matter.

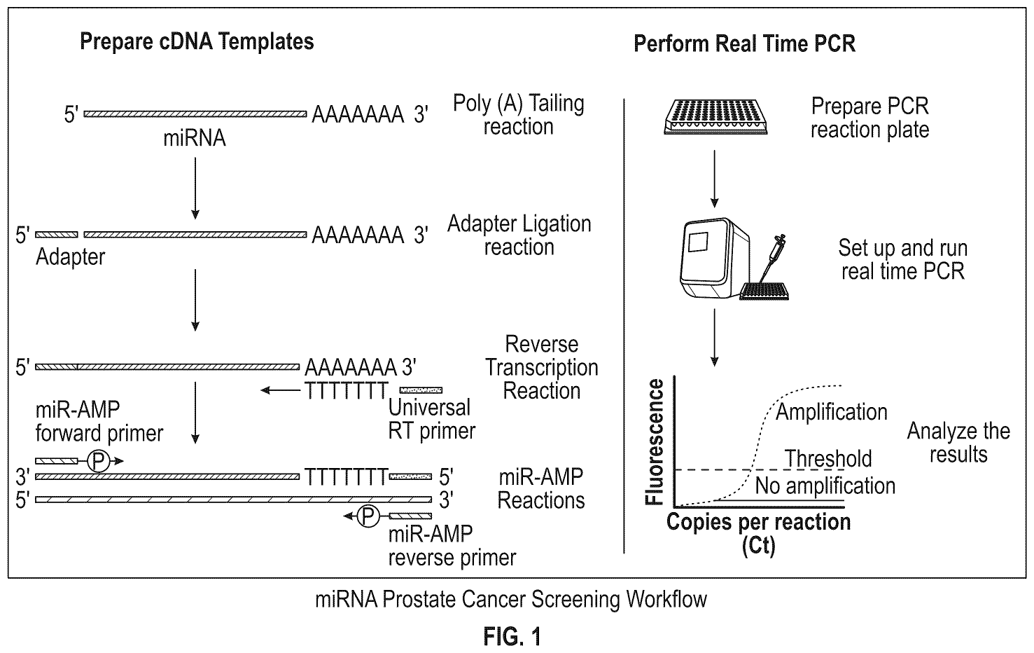

In an embodiment, miRNA detection and quantification involves extraction of cell-free DNA from urine specimens, preparation of complementary DNA (cDNA) templates, quantification via real-time PCR amplification and data analysis using the expression suite software from Thermo Fisher Scientific Inc. and a customized list of miRNA targets to assess miRNA levels, see FIG. 1.

The method comprises:
1. Providing and optional storage of unaltered urine sample
2. miRNA extraction from the self-collected urine sample
3. Detection of miRNA levels using RT-qPCR (Cycle Threshold)
4. Determining a positive or negative result for urological cancer based on the levels of specific miRNA.

In one aspect the method relates to detection of levels of miRNAs in urine samples of cis-gender, non-binary, and trans-gender patients.

In another aspect the means for extracting total RNA comprises RNA extraction reagents and equipment.

In one aspect the method can detect a group of miRNAs including hsa-let-7b5p, has-miR-26b5p, has-miR-1455p, has-miR-4253p, has-miR-1955p, has-miR-203a3p, has-miR-30c5p, has-miR-30a3p.

In one embodiment, the miRNA analysis is performed using reverse transcription quantitative polymerase chain reaction (RT-qPCR). In some embodiments, the miRNA analysis is performed using next-generation sequencing (NGS). In another embodiment the miRNA analysis is performed using microarray analysis.

In another aspect the test is non-invasive, easily accessible, and cost-effective screening test capable of accurately detecting urological cancer such as that of prostate, urinary bladder, kidney, renal pelvis, and testis.

In one aspect the method provides a screening test for detecting urological cancer in transgender women, and non-binary people. Consequently, this generates avenues for devising protocols to manage positive test outcomes, encompassing the implementation of suitable medical interventions and mitigating healthcare expenses.

In another embodiment the method provides a gender-neutral urine-based test for, e.g., cis-gender, non-binary, and transgender patients allowing them to self-sample their urine sample at-home or in private to ship back to the laboratory for urogenital cancer testing via mail.

In certain embodiments of the invention provide different miRNAs that are present differentially in urine sample of patients having urological cancer, or an individual having increased risk of having urological cancer, in comparison to the healthy individuals or the one with low risk of developing urological cancer.

In one embodiment the individual having urological cancer or high risk of developing urological cancer has overexpression or low expression of one are multiple miRNAs including hsa-let-7b5p, has-miR-26b5p, has-miR-1455p, has-miR-4253p, has-miR-1955p, has-miR-203a3p, has-miR-30c5p, has-miR-30a3p in their urine sample.

In one embodiment the miRNAs described here can be used to distinguish individuals having urological cancer from those having no urological cancer, or to distinguish individuals having high risk of developing urological cancer from individuals having low risk of urological cancer.

In one embodiment the sample to be used for the detection of miRNAs can be body fluid including urine, whole blood, plasma, serum, or saliva.

In one embodiment the method of detecting miRNAs in samples can be quantitative Polymerase Chain Reaction (qPCR), next generation sequencing or mass spectrophotometry.

In one embodiment the panel of miRNAs include but not limited to hsa-let-7b5p, has-miR-26b5p, has-miR-1455p, has-miR-4253p, has-miR-1955p, has-miR-203a3p, has-miR-30c5p, has-miR-30a3p.

According to one aspect, the present invention provides a method for screening urological cancer in non-binary individuals having urological cancer or having high risk of developing urological cancer.

In one embodiment the method comprises of determining the expression levels of plurality of miRNAs that are selected from a group of miRNAs including hsa-let-7b5p, has-miR-26b5p, has-miR-1455p, has-miR-4253p, has-miR-1955p, has-miR-203a3p, has-miR-30c5p, has-miR-30a3p or combinations thereof in a urine sample obtained from subject.

According to one embodiment the overexpression of plurality of miRNAs in a biological sample such as urine compared to control values is indicative of urological cancer or high risk of developing urological cancer in the said subject. The examples provided herein are intended to be merely exemplary, and those skilled in the art will recognize, or will be able to ascertain using no more than routine experimentation, numerous equivalents of specific samples, materials, and procedures. All such equivalents are considered to be within the scope of the invention and are encompassed by the appended claims.

### Examples

FIG. 1 provides a process overview of an exemplary embodiment of the invention which includes:

### Sample Processing and Extraction

Preparing cDNA templates.

Performing real-time PCR.

Expression data analysis.

### Example 1 Materials and Equipment

Provided in Table 1 below is a list of materials used in an exemplary embodiment of the invention and provided in Table 2 is a list of equipment used in an exemplary embodiment of the invention.

TABLE 1
Reagents Details

| | | Storage | |
|---|---|---|---|
| Manufacturer | Reagent | Conditions | |
| Life Technologies Corporation | mirVana ™ miRNA | −200° | ^{C.} |
| | Isolation Kit | | |
| Life Technologies Corporation | 10X Poly(A) Buffer | −200° | ^{C.} |
| Life Technologies Corporation | ATP, 10 mM | −200° | ^{C.} |
| Life Technologies Corporation | Poly(A) Enzyme, 5 U/μL | −200° | ^{C.} |
| Life Technologies Corporation | 5X DNA Ligase Buffer | −200° | ^{C.} |
| Life Technologies Corporation | RNA Ligase, 10 U/μL | −200° | ^{C.} |
| Life Technologies Corporation | 50% PEG 8000 | −200° | ^{C.} |
| Life Technologies Corporation | 25X Ligation Adaptor | −200° | ^{C.} |
| Life Technologies Corporation | 10X RT Enzyme Mix | −200° | ^{C.} |
| Life Technologies Corporation | 5X RT Buffer | −200° | ^{C.} |
| Life Technologies Corporation | 20X Universal RT | −200° | ^{C.} |
| | Primer | | |
| Life Technologies Corporation | dNTP Mix, 100 mM | −200° | ^{C.} |
| Life Technologies Corporation | 20X miR-Amp Primer | −200° | ^{C.} |
| | Mix | | |
| Life Technologies Corporation | 2X miR-Amp Master | 40° | ^{C.} |
| | Mix | | |

TABLE 2
Equipment Details

| Instrument | Supplier | Serial No. |
|---|---|---|
| AutoPure-96 | Allsheng | 180058575-002827 |
| Veriti 96-Well Thermocycler | Thermofisher | 2990242084 |
| MiniseqQuantStudio ™ 12K | Thermofisher | |
| Flex Real-Time PCR System | | |

Endogenous control Hsa-miR-191-5p was purchased from the Life Technologies Corporation and was used for validation and as endogenous control with each clinical run.

### Example 1.1: Preparation for Extraction

Samples were entered into a sample tracking database by logging accession numbers into a running excel sheet for miR-ProstateDx panel runs. Deep-well plates were planned according to sample number, extraction controls, and non-template controls. Specimens that were fully processed were stored in a refrigerator. An 80% ethanol solution was prepared so that there was enough for 1.5 mL per reaction with 10% overage using 100% absolute ethanol and Nuclease-free water. Extraction was performed by thawing and mixing all the samples. 100% Isopropanol was used to Wash the concentrates twice. A heating block was set to 65° C. and 1% 2-mercaptoethanol was added to Lysis Buffer just before use. Sufficient Lysis Binding Mix was prepared according to the below Table 3. 250 μL of urine sample was combined with 200 μL of Lysis Binding Mix in a KingFisher™ 96 Deep-Well Plate. The plate was covered and then shaken. 480 μL of isopropanol was added to each sample and the samples were then processed on the instrument.

TABLE 3

| | |
|---|---|
| Lysis Buffer | 198 μL |
| 2-Mercaptoethanol | 2 μL |
| Total | |

### Example 1.2: Plate Setup and Configuration

While the samples were incubating, the Wash, DNase, Elution, and Tip Comb Plates were set up outside the instrument as described in the following table:

TABLE 4
Plate setup

| | | Plate | | Volume |
|---|---|---|---|---|
| Step | Name | Position | Reagent | (uL) |
| 1 | Wash plate1 | 2 | Wash Solution 1 | 150 uL |
| 2 | Wash plate 2 | 3 | Wash Solution 2 | 150 uL |
| 3 | DNase Plate | 4 | TURBO DNase ™ | 50 uL |
| | | | Solution | |
| 4 | Wash plate 3 | 5 | Wash Solution 2 | 150 uL |
| 5 | Wash plate 4 | 6 | Wash Solution 2 | 150 uL |
| 6 | Elution Plate | 7 | Elution Buffer | 50 uL |
| 7 | Tip Comb | 8 | | |

The instrument was confirmed to be equipped with a deep well magnetic head. The run was initiated and then the prepared processing plates were inserted into their designated slots. The sample plate was placed in position (with lysate, isopropanol and binding beads included) and approximately 30-35 minutes later the DNase plate was removed from the instrument. 50 μL of rebinding buffer and 100 μL of isopropanol was added to each sample well. Care was takent to avoid over-drying any beads retained on the Tip Comb. The DNase Plate was then reinserted into the instrument and the run was started. When the run finished, the late was carefully sealed and stored. The elution plate may be stored on ice for up to 8 hours, or at −20° C. or −80° C. for extended preservation.

### Example 1.3: MiRNA Expression Workflow—Performing the Poly(A) Tailing Reaction

The samples and cDNA synthesis reagents were thawed on ice, then gently vortexed to ensure all components are well mixed. The tubes were briefly centrifuged to bring the contents to the bottom and eliminate any air bubbles. Enough Poly(A) Reaction Mix in 1.5-mL microcentrifuge tubes were prepared for the total number of reactions required by vortexing the Poly(A) Reaction Mix to ensure it was thoroughly blended, then briefly centrifuging it to collect the contents and to remove any air bubbles. 2 μL of the sample was dispensed into each well of a reaction plate or individual reaction tube, followed by 3 μL of the Poly(A) Reaction Mix. The reaction plate or tubes were securely sealed, then briefly vortexed to mix the contents evenly. Again the tubes were centrifuged to collect contents and remove air bubbles. The reaction plates or tubes were then inserted into a thermal cycler and the incubation was run using the parameters outlined below in Table 5:

TABLE 5
PCR Parameters

| Step | Temperature | Time |
|---|---|---|
| Polyadenylation | 37° C. | 45 minutes |
| Stop reaction | 65° C. | 10 minutes |
| Hold | 4° C. | Hold |

### Example 1.4: Performing the Adaptor Ligation Reaction

Using a 1.5-mL microcentrifuge tube, the necessary volume of Ligation Reaction Mix was prepared to accommodate the total number of reactions, following Table 6 below:

TABLE 6
Components of Ligation Reaction Mix

| Component | 1 Rxn |
|---|---|
| 5X DNA Ligase Buffer | 3 μL |
| 50% PEG 8000[2] | 4.5 μL |
| 25X Ligation Adaptor | 0.6 μL |
| RNA Ligase | 1.5 μL |
| RNase-free water | 0.4 μL |
| Total Ligation Reaction Mix volume | 10 μL |

The Ligation Reaction Mix was vortexed thoroughly to ensure it was well combined, then briefly centrifuged to bring down the contents and eliminate air bubbles. 10 μL of the Ligation Reaction Mix was dispensed into each well or tube that contained the poly(A) tailing reaction product. The reaction plates or tubes were sealed, then briefly vortexed or shaken (e.g., 1,900 rpm for 1 minute using an Eppendorf™ MixMate™) to mix the contents completely. The plates or tubes were then briefly centrifuged to collect the contents at the bottom and remove air bubbles. The reaction plate or tubes were loaded into a thermal cycler and run with the following thermal profile in Table 7.

TABLE 7

| Step | Temperature | Time |
|---|---|---|
| Ligation | 16° C. | 60 minutes |
| Hold | 4° C. | Hold |

### Example 1.5 Performing (without Delay) the Reverse Transcription (RT) Reaction

In a 1.5-mL microcentrifuge tube, an adequate volume of RT Reaction Mix was prepared based on the number of reactions needed, using the following Table 8 as a guide:

TABLE 8
Components of RT Reaction Mix

| Component | 1 Rxn |
|---|---|
| 5X RT Buffer | 6 μL |
| dNTP Mix (25 mM each) | 1.2 μL |
| 20X Universal RT Primer | 1.5 μL |
| 10X RT Enzyme Mix | 3 μL |
| RNase-free water | 3.3 μL |
| Total RT Reaction Mix volume | 15 μL |

The RT Reaction Mix was vortexed thoroughly to ensure complete mixing of the components, then briefly centrifuged to collect the contents at the bottom and remove any air bubbles. 15 μL of the RT Reaction Mix was transferred into each well of the reaction plate or each reaction tube that contained the adaptor ligation reaction product. The reaction plate or tubes were sealed, then vortexed briefly to ensure thorough mixing of the contents. The reaction plate or tubes were then briefly centrifuged to bring the contents to the bottom and eliminate any remaining air bubbles. The reaction plate or tubes were placed into a thermal cycler and incubated using the following thermal profile in Table 9 below (after this reaction, the samples may be stored at −20° C. for up to 2 months for future use).

TABLE 9
PCR Thermal Profile

| Step | Temperature | Time |
|---|---|---|
| Reverse transcription | 42° C. | 15 minutes |
| Stop reaction | 85° C. | 5 minutes |
| Hold | 4° C. | Hold |

### Example 1.6 Performing the miR-Amp Reaction

In a 1.5-mL microcentrifuge tube, the necessary amount of miR-Amp Reaction Mix was prepared based on the total number of reactions required, using Table 10 below:

TABLE 10
Components of miR-Amp Reaction Mix

| Component | 1 Rxn |
|---|---|
| 2X miR-Amp Master Mix | 25 μL |
| 20X miR-Amp Primer Mix | 2.5 μL |
| RNase-free water | 17.5 μL |
| Total miR-Amp Reaction Mix volume | 45 μL |

The miR-Amp Reaction Mix was vortexed thoroughly to ensure all components are well mixed, then briefly centrifuged to collect the contents at the bottom and remove any air bubbles. 45 μL of the miR-Amp Reaction Mix was transferred into each well of a new reaction plate or into each reaction tube. 5 μL of the RT reaction product was added to each well or reaction tube. The reaction plate or tubes were sealed, then briefly vortexed to ensure thorough mixing of the contents. The reaction plate or tubes were briefly cetributed to bring the contents to the bottom and eliminate any air bubbles. The reaction plate or tubes were placed into a thermal cycler and incubated using the following thermal profile, with maximum ramp speed, and standard cycling settings given in Table 11 below. Teal-time PCR was performed as described below. The undiluted miR-Amp reaction product at −20° C. for up to 2 months for future use.

TABLE 11
PCR Thermal Profile

| Step | Temperature | Time | Cycles |
|---|---|---|---|
| Enzyme activation | 95° C. | 5 minutes | 1 |
| Denature | 95° C. | 3 seconds | 14 |
| Anneal/Extend | 60° C. | 30 seconds | |
| Stop reaction | 99° C. | 10 minutes | 1 |
| Hold | 4° C. | Hold | 1 |

### Example 1.7: Preparation of PCR Reaction Plate

The assays were thawed on ice, gently vortexed to ensure thorough mixing, then briefly centrifuged to collect the contents and remove any air bubbles. A 1:10 dilution of the cDNA template was prepared. A bottle of TaqMan® Fast Advanced Master Mix was shaken to mix the contents well, avoiding inversion of the bottle. In a 1.5-mL microcentrifuge tube, the required amount of PCR Reaction Mix was prepared based on the number of reactions needed, following Table 12 below:

TABLE 12
Components of PCR Reaction Mix

| Component | 1 Rxn |
|---|---|
| TaqMan ® Fast Advanced Master Mix (2X) | 10 μL |
| TaqMan ® Advanced miRNA Assay (20X) | 1 μL |
| RNase-free water | 4 μL |
| Total PCR Reaction Mix volume | 15 μL |

The PCR Reaction Mix was vortexed thoroughly to ensure complete mixing, then briefly centrifuge to collect the contents at the bottom and eliminate any air bubbles. 15 μL of the PCR Reaction Mix was transferred into each well of the PCR reaction plate. 5 μL of the diluted cDNA template was added into each well of the plate. The reaction plate was sealted with an adhesive cover, then briefly vortexed to ensure thorough mixing of the contents followed by centrifuging to collect the contents at the bottom.

### Example 1.8: Setting Up and Running the Real-Time PCR Instrument

Refer to the relevant instrument user guide for detailed instructions on how to program the thermal-cycling conditions or run the plate. The following thermal profiles were optimized for use with TaqMan® Fast Advanced Master Mix and are suitable for both Fast and Standard reaction plates, as well as corresponding instrument block configurations. The reaction plate was loaded into the real-time PCR instrument. The appropriate experiment settings and PCR thermal cycling conditions were set and the run was started, see Table 13 below.

TABLE 13
PCR Thermal Profile

| Step | Temperature | Time | Cycles |
|---|---|---|---|
| Enzyme activation | 95° C. | 20 seconds | 1 |
| Denature | 95° C. | 1 second | 40 |
| Anneal/Extend | 60° C. | 20 econds | |

### Example 2: Validation Plan and Summary for Prostate Cancer miRNA Panel

The validation process is conducted by two trained technologists over a 4-week period, utilizing a total of 30 human samples, alongside a negative extraction control and a non-template control. To assess the performance characteristics of the assay, both accuracy and precision studies are executed. Precision studies are performed through both intra-assay (within a single run) and inter-assay (across multiple runs conducted on separate days) analyses to evaluate the reproducibility of results. The intra-assay study consists of 30 samples, each run in triplicates, while the inter-assay studies include three runs, each conducted in single replicates, to assess the assay's reproducibility across different days. The accuracy of the assay is evaluated by comparing the observed expression calls to the expected results derived from reference materials. Data analysis is performed using ExpressionSuite Software v1.3. Clinically positive samples have been previously validated through prostate-specific antigen (PSA) blood levels and, in certain cases, through histopathological analysis of prostate tissue. Validation objectives are to (1) establish the analytical sensitivity and specificity of the miRNA expression assay across both inter- and intra-run conditions, (2) confirm the assay's accuracy in distinguishing between high and low miRNA expression levels in clinical samples, and (3) evaluate the reproducibility and reliability of the assay under routine laboratory conditions.

### Example 2.1: Validation Workflow

This validation plan outlined the procedures and methodologies used to assess the performance characteristics of the Prostate Cancer miRNA Panel, utilizing the TaqMan™ Advanced miRNA Assay system. The validation process aimed to ensure the assay's accuracy, precision, and reliability for clinical applications. A total of 30 human clinical samples, including prostate cancer-positive and negative controls, were collected. RNA was extracted using a validated method that preserved small RNAs, ensuring the integrity and quality of the samples. The extracted RNA was quantified and assessed for purity to meet the assay's input requirements. The validation assessed the assay's accuracy, precision, and reproducibility:

Accuracy: Final expression calls were compared with expected results from reference materials to determine the assay's ability to correctly identify miRNA expression levels.

Precision: Both intra-assay (within the same run) and inter-assay (across different runs) precision were evaluated by analyzing multiple replicates of the same samples to assess variability.

Reproducibility: The assay's performance was tested across different days and operators to ensure consistent results.

### Example 2.2 Data Analysis and Software Utilization

Expression data was analyzed using ExpressionSuite Software v1.3, which provided comprehensive tools for miRNA quantification and analysis. This software facilitated the assessment of assay performance metrics and supported the validation process. Clinically positive samples had been previously confirmed through blood PSA levels and, in some cases, prostate tissue histopathology. Negative extraction controls and no-template controls were included to assess potential contamination and ensure assay specificity. The performance of the assay was correlated with clinical outcomes to establish its diagnostic utility.

### Example 2.3: Regulatory Compliance

This validation plan was designed to meet the requirements for submission to regulatory bodies such as CLIA. All procedures adhered to Good Laboratory Practices (GLP) and relevant regulatory guidelines to ensure the assay's suitability for clinical use. Upon successful completion of the validation, the Prostate Cancer miRNA Panel was deemed ready for implementation in clinical diagnostics, providing a reliable tool for the screening and monitoring of prostate cancer.

### Example 2.4 Specimens Included and Usage

A total of 30 human samples were used in the validation study, accompanied by two negative controls: a negative extraction control and a no-template control (Table 14 below). These samples formed the basis for evaluating the assay's performance characteristics, specifically its accuracy and precision. To assess precision, both intra-assay and inter-assay studies were conducted to evaluate reproducibility under different conditions. The intra-assay precision study involved testing all 10 human samples in triplicate within a single analytical run to measure consistency within the same run. For inter-assay precision, the same set of 30 samples was tested across three separate runs conducted on different days, using single replicates, to assess day-to-day variability. Together, these analyses determined the assay's reliability and robustness over time and under routine laboratory conditions.

TABLE 14
List of Patient Samples Used in Clinical Validation

Clinically positive patient 1
Clinically positive patient 2
Clinically positive patient 3
Clinically positive patient 4
Clinically positive patient 5
Clinically positive patient 6
Clinically positive patient 7
Clinically positive patient 8
Clinically positive patient 9
Clinically positive patient 10
Clinically positive patient 11
Clinically positive patient 12
Clinically positive patient 13
Clinically positive patient 14
Clinically positive patient 15
Clinically positive patient 16
Clinically positive patient 17
Clinically positive patient 18
Clinically positive patient 19
Clinically positive patient 20
Clinically positive patient 21
Clinically positive patient 22
Clinically positive patient 23
Clinically positive patient 24
Clinically positive patient 25
Clinically Negative patient 1
Clinically Negative patient 2
Clinically Negative patient 3
Clinically Negative patient 4
Clinically Negative patient 5
ENTC
NTC

Four sequencing runs (3 inter-runs and 1 intra-run) were conducted to evaluate the performance characteristics of the miRNA expression assay for prostate cancer detection. A total of 32 samples were included in the study, comprising 25 clinically confirmed prostate cancer-positive samples, 5 negative samples, one no-template control (NTC), and one extraction no-template control (ENTC). The 25 positive and 5 negative samples, along with their replicates, were sequenced to assess the assay's accuracy, sensitivity, and reproducibility. The five negative samples served as non-cancer controls to support specificity analysis, while the NTC and ENTC were included to monitor for contamination during extraction and amplification. Known control samples were not used in this study, as performance metrics were derived from clinical expression patterns across validated miRNA targets relevant to prostate cancer. Expression profiles from positive cases were compared against negative and background controls to determine the assay's ability to reliably differentiate disease presence from normal expression levels. Test samples: 20 new samples were sequenced and used for studying the reproducibility of results.

During validation, poorly performing miRNA assays will be identified through systematic analysis of key performance metrics such as low amplification efficiency, high variability across replicates, inconsistent detection across runs and poor linearity. Common causes of poor assay performance include suboptimal primer or probe design, low endogenous expression of target miRNAs, RNA degradation, and non-specific amplification. Identifying these issues early allows for assay refinement to ensure reliability and compliance with standards.

### Example 2.5: Validation Results—Inter Run miRNA Expression Results

Three inter-assay runs were performed using a total of 32 samples, including one no-template control (NTC) and one extraction no-template control (ENTC). For each sample, the Ct values obtained from the three independent runs were averaged to calculate a mean Ct value for each miRNA target, allowing for consistent assessment across runs. Ct cutoff thresholds were then established based on the results from the negative samples and control specimens (Table 15). A “2 out of 3 passing” criterion was applied meaning a sample was considered positive if at least two out of the three runs yielded Ct values below the established threshold for that miRNA target. Ct cutoff thresholds were determined based on the Ct distributions observed in the negative samples and control specimens. Of the 25 clinically confirmed positive samples, 24 met the criteria for a positive call, while one sample did not meet the cutoff in at least two runs and was reported as a false negative. All five negative samples were correctly identified as true negatives. Both the NTC and ENTC showed no amplification across all runs, confirming the absence of contamination. Detailed results are summarized in Table below.

TABLE 15
Ct values across the three inter validation runs
miRNA Targets
Ct value

| | | | | | | Endogenous |
|---|---|---|---|---|---|---|
| | Hsa-let- | Hsa-miR- | Hsa-miR- | Hsa-let- | Hsa-miR- | control |
| Samples | 7b-5p | 203a-3p | 148a-3p | 7a-5p | 30A-3p | Hsa-miR-191-5p |
| Patient 1 | 32.18633333 | 31.806 | 30.30233333 | 27.634 | 28.64766667 | 27.40333333 |
| Patient 2 | 36.1845 | 33.577 | 35.6245 | 30.956 | 33.093 | 32.82366667 |
| Patient 3 | 32.70666667 | 29.3365 | 30.27466667 | 27.706 | 28.42866667 | 26.1965 |
| Patient 4 | 34.59833333 | 30.846 | 35.5855 | 29.54833333 | 32.26766667 | 31.613 |
| Patient 5 | 32.76033333 | 29.1925 | 30.305 | 28.708 | 28.66133333 | 26.081 |
| Patient 6 | 34.36833333 | 30.593 | 36.691 | 29.36866667 | 32.27933333 | 31.658 |
| Patient 7 | 32.65966667 | 29.574 | 30.28266667 | 27.77033333 | 28.55233333 | 27.34233333 |
| Patient 8 | 35.696 | 32.702 | 36.004 | 29.98433333 | 32.48166667 | 32.039 |
| Patient 9 | 32.972 | 29.3625 | 30.70433333 | 27.895 | 28.66066667 | 27.15666667 |
| Patient 10 | 36.22666667 | 33.683 | 37.79 | 30.40366667 | 33.201 | 32.37766667 |
| Patient 11 | 33.27166667 | 29.252 | 30.519 | 28.09866667 | 28.45666667 | 25.86866667 |
| Patient 12 | 36.39933333 | 34.183 | 35.5 | 31.07233333 | 33.14733333 | 33.289 |
| Patient 13 | 33.00133333 | 29.364 | 30.49866667 | 28.03933333 | 28.33766667 | 27.28566667 |
| Patient 14 | 36.55066667 | 34.111 | 35.747 | 30.93033333 | 33.96066667 | 32.90533333 |
| Patient 15 | 32.731 | 31.836 | 30.794 | 27.88633333 | 28.82066667 | 27.45 |
| ENTC | NA | NA | 37.188 | 33.9985 | 35.1825 | NA |
| Patient 16 | 31.49233333 | 27.376 | 28.41933333 | 26.82033333 | 27.86533333 | 26.12766667 |
| Patient 17 | 31.29333333 | 31.18333333 | 28.30466667 | 26.43566667 | 27.62133333 | 25.751 |
| Patient 18 | 31.30033333 | 26.8685 | 27.85133333 | 26.469 | 27.61533333 | 25.71633333 |
| Patient 19 | 31.756 | 27.6175 | 28.64866667 | 27.02833333 | 27.96033333 | 26.32066667 |
| Patient 20 | 29.88866667 | 30.21766667 | 27.54033333 | 26.043 | 27.04733333 | 25.38633333 |
| Patient 21 | 37.845 | 32.8245 | 34.63 | 31.8185 | 33.18033333 | 30.28233333 |
| Patient 22 | 37.8305 | 32.696 | NA | 33.2195 | 32.9975 | 32.65466667 |
| Patient 23 | 35.02 | 31.6555 | 27.886 | 31.1685 | 32.90633333 | 29.24366667 |
| Patient 24 | 37.21 | 32.142 | NA | 31.408 | 34.639 | 32.19466667 |
| Patient 25 | 36.86866667 | 31.8905 | 31.329 | 30.591 | 32.29833333 | 29.27733333 |
| Patient26 | 37.621 | 31.406 | NA | 32.3525 | 34.32633333 | 31.69833333 |
| Patient 27 | 37.73266667 | 32.759 | 32.379 | 31.573 | 32.921 | 30.04433333 |
| Patient 28 | 37.334 | 32.176 | NA | 31.743 | 33.008 | 32.62366667 |
| Patient 29 | 37.4695 | 32.915 | 36.101 | 32.0795 | 33.864 | 30.46866667 |
| Patient 30 | 37.769 | NA | NA | NA | NA | 32.94433333 |
| NTC | NA | NA | NA | NA | NA | NA |

TABLE 16
Ct Thresholds for miRNA Targets

| miRNA Target | Ct Cutoff |
|---|---|
| Hsa-let-7b-5p | 37 |
| Hsa-miR-203a-3p | 32 |
| Hsa-miR-148a-3p | 38 |
| Hsa-let-7a-5p | 32 |
| Hsa-miR-30A-3p | 34 |
| Hsa-miR-191-5p (Control) | 33 |

TABLE 17
Positive and negative calls based on the expression profile of each sample

| | miRNA Targets | | | | | Endogenous |
|---|---|---|---|---|---|---|
| | Hsa-let- | Hsa-miR- | Hsa-miR- | Hsa-let- | Hsa-miR- | control |
| Samples | 7b-5p | 203a-3p | 148a-3p | 7a-5p | 30A-3p | Hsa-miR-191-5p |

Patient 1
Patient 2
Patient 3
Patient 4
Patient 5
Patient 6
Patient 7
Patient 8
Patient 9
Patient 10
Patient 11
Patient 12
Patient 13
Patient 14
Patient 15
ENTC
Patient 16
Patient 17
Patient 18
Patient 19
Patient 20
Patient 21
Patient 22
Patient 23
Patient 24
Patient 25
Patient26
Patient 27
Patient 28
Patient 29
Patient 30
NTC

TABLE 18
Ct Values for Target miRNAs by Patient Sample

| | Hsa- | Hsa- | Hsa- | Hsa- | Hsa- | Hsa- |
|---|---|---|---|---|---|---|
| | let- | miR- | miR- | let- | miR- | miR- |
| | 7b-5p | 203a-3p | 148a-3p | 7a-5p | 30A-3p | 191-5p |
| Patient 1 | 33.968 | 32.317 | 33.978 | 30.029 | 32.298 | 29.665 |
| Patient 2 | 31.651 | 29.71 | 31.654 | 27.228 | 29.281 | 26.953 |
| Patient 3 | 34.732 | 31.849 | 35.155 | 29.66 | 32.749 | 31.635 |
| Patient 4 | 33.919 | 31.759 | 35.055 | 29.306 | 31.941 | 31.647 |
| Patient 5 | 28.558 | 25.766 | 24.328 | 24.28 | 26.101 | 22.836 |
| Patient 6 | 27.859 | 26.029 | 24.374 | 24.266 | 25.963 | 22.307 |
| Patient 7 | 35.303 | 30.919 | 30.41 | 29.526 | 32.743 | 28.29 |
| Patient 8 | 34.211 | 30.682 | 30.152 | 29.002 | 32.62 | 28.397 |
| Patient 9 | 37.965 | NA | NA | 33.264 | NA | 31.34 |
| Patient 10 | 37.718 | NA | NA | 33.103 | NA | 31.331 |
| ENTC | NA | NA | NA | NA | NA | NA |
| NTC | NA | NA | NA | NA | NA | NA |

TABLE 19
Expression Calls for Each miRNA
Target per Based on Ct Thresholds

| | | | | | |
|---|---|---|---|---|---|
| Hsa- | Hsa- | Hsa- | Hsa- | Hsa- | Hsa- |
| let- | miR- | miR- | let- | miR- | miR- |
| 7b-5p | 203a-3p | 148a-3p | 7a-5p | 30A-3p | 191-5p |

Patient 1
Patient 2
Patient 3
Patient 4
Patient 5
Patient 6
Patient 7
Patient 8
Patient 9
Patient 10
ENTC
NTC

TABLE 20
Overall Sensitivity

| Sensitivity | Both Inter and Intra runs |
|---|---|
| Total number of miRNA calls | 264 |
| High miRNA expression | 185 |
| (true positives. TP) | |
| Low miRNA expression | 13 |
| (false negatives. FN) | |
| Sensitivity {[TP/(TP + | 93.8% |
| FN)]*100} | |

TABLE 21
Overall Specificity

| Specificity | Both Inter and Intra runs |
|---|---|
| Total number of miRNA calls | 264 |
| High miRNA expression | 1 |
| (False positives. TP) | |
| Low expression miRNA | 65 |
| (True negatives. TN) | |
| Specificity {100% × | 98.4% |
| TN/(FP + TN)} | |

TABLE 22
Overall Accuracy

| Accuracy | Both Inter and Intra runs |
|---|---|
| Total number of miRNA calls | 264 |
| High expression miRNA | 185 |
| (true positives. TP) | |
| Low expression miRNA | 65 |
| (True negatives. TN) | |
| Sensitivity {100% × | 94.6% |
| (TP + TN)/Total} | |

### Test Reproducibility

Based on the inter-run reproducibility data, the miRNA expression assay demonstrated high consistency across three independent runs. Of the 30 patient samples evaluated (excluding the NTC and ENTC), 29 showed 100% reproducibility, with each sample producing the same result—either true positive (TP) or true negative (TN)—in all three runs. One sample (Patient 29) yielded two true positives and one false negative across the runs, resulting in a reproducibility rate of 66.6% for that individual. Overall, the assay achieved 100% reproducibility in 96.7% of the samples (29 out of 30), indicating strong inter-run reliability under routine testing conditions. Both the NTC and ENTC consistently returned true negative results across all runs, further supporting the assay's robustness and lack of cross-contamination. Intra-run reproducibility was similarly evaluated by running each sample in triplicate within a single run. All patient samples, including controls, demonstrated 100% concordance across replicates. Specifically, all positive samples returned consistent true positive results, while all negative samples—including both the ENTC and NTC—consistently tested true negative. No false positives or false negatives were observed in any of the replicates, confirming excellent assay precision within a single analytical run. Together, these results affirm the high reproducibility and analytical stability of the assay, supporting its suitability for diagnostic use.

TABLE 23
Intra-run Assay Reproducibility

| miRNAs | Replicate | Replicate | Replicate | #True | #Expected | |
|---|---|---|---|---|---|---|
| assays | 1 | 2 | 3 | calls | calls | Reproducibility |
| Hsa-let-7b-5p | 10 | 10 | 12 | 32 | 32 | 100% |
| Hsa-miR-203a-3p | 10 | 10 | 12 | 32 | 32 | 100% |
| Hsa-miR-148a-3p | 10 | 10 | 12 | 32 | 32 | 100% |
| Hsa-let-7a-5p | 10 | 10 | 12 | 32 | 32 | 100% |
| Hsa-miR-30A-3p | 10 | 10 | 12 | 32 | 32 | 100% |
| Hsa-miR-191-5p | 10 | 10 | 11 | 32 | 31 | 96.8% |
| Endogenous | | | | | | |
| control | | | | | | |

TABLE 24
Intra-run Patient Reproducibility

| Patient | Run | Run | Run | True | True | False | False | |
|---|---|---|---|---|---|---|---|---|
| Samples | 1 | 2 | 3 | Positive | Negative | Positive | Negative | Reproducibility |
| Patient 1 | TP | TP | TP | 3 | 0 | 0 | 0 | 100% |
| Patient 2 | TP | TP | TP | 3 | 0 | 0 | 0 | 100% |
| Patient 3 | TP | TP | TP | 3 | 0 | 0 | 0 | 100% |
| Patient 4 | TP | TP | TP | 3 | 0 | 0 | 0 | 100% |
| Patient 5 | TP | TP | TP | 3 | 0 | 0 | 0 | 100% |
| Patient 6 | TP | TP | TP | 3 | 0 | 0 | 0 | 100% |
| Patient 7 | TP | TP | TP | 3 | 0 | 0 | 0 | 100% |
| Patient 8 | TP | TP | TP | 3 | 0 | 0 | 0 | 100% |
| Patient 9 | TP | TP | TP | 3 | 0 | 0 | 0 | 100% |
| Patient 10 | TP | TP | TP | 3 | 0 | 0 | 0 | 100% |
| Patient 11 | TP | TP | TP | 3 | 0 | 0 | 0 | 100% |
| Patient 12 | TP | TP | TP | 3 | 0 | 0 | 0 | 100% |
| Patient 13 | TP | TP | TP | 3 | 0 | 0 | 0 | 100% |
| Patient 14 | TP | TP | TP | 3 | 0 | 0 | 0 | 100% |
| Patient 15 | TP | TP | TP | 3 | 0 | 0 | 0 | 100% |
| ENTC | TN | TN | TN | 0 | 3 | 0 | 0 | 100% |
| Patient 16 | TP | TP | TP | 3 | 0 | 0 | 0 | 100% |
| Patient 17 | TP | TP | TP | 3 | 0 | 0 | 0 | 100% |
| Patient 18 | TP | TP | TP | 3 | 0 | 0 | 0 | 100% |
| Patient 19 | TP | TP | TP | 3 | 0 | 0 | 0 | 100% |
| Patient 20 | TP | TP | TP | 3 | 0 | 0 | 0 | 100% |
| Patient 21 | TP | TP | TP | 3 | 0 | 0 | 0 | 100% |
| Patient 22 | TN | TN | TN | 0 | 3 | 0 | 0 | 100% |
| Patient 23 | TP | TP | TP | 3 | 0 | 0 | 0 | 100% |
| Patient 24 | TN | TN | TN | 0 | 3 | 0 | 0 | 100% |
| Patient 25 | TP | TP | TP | 3 | 0 | 0 | 0 | 100% |
| Patient26 | TN | TN | TN | 0 | 3 | 0 | 0 | 100% |
| Patient 27 | TP | TP | TP | 3 | 0 | 0 | 0 | 100% |
| Patient 28 | TN | TN | TN | 0 | 3 | 0 | 0 | 100% |
| Patient 29 | TP | TP | FN | 2 | 0 | 0 | 1 | 66.6% |
| Patient 30 | TN | TN | TN | 0 | 3 | 0 | 0 | 100% |
| NTC | TN | TN | TN | 0 | 3 | 0 | 0 | 100% |

TABLE 25
Intra-run Patient Reproducibility

| Patient | Replicate | Replicate | Replicate | True | True | False | False | |
|---|---|---|---|---|---|---|---|---|
| Samples | 1 | 2 | 3 | Positive | Negative | Positive | Negative | Reproducibility |
| Patient 1 | TP | TP | TP | 3 | 0 | 0 | 0 | 100% |
| Patient 2 | TP | TP | TP | 3 | 0 | 0 | 0 | 100% |
| Patient 3 | TP | TP | TP | 3 | 0 | 0 | 0 | 100% |
| Patient 4 | TP | TP | TP | 3 | 0 | 0 | 0 | 100% |
| Patient 5 | TP | TP | TP | 3 | 0 | 0 | 0 | 100% |
| Patient 6 | TP | TP | TP | 3 | 0 | 0 | 0 | 100% |
| Patient 7 | TP | TP | TP | 3 | 0 | 0 | 0 | 100% |
| Patient 8 | TP | TP | TP | 3 | 0 | 0 | 0 | 100% |
| Patient 9 | TN | TN | TN | 0 | 3 | 0 | 0 | 100% |
| Patient 10 | TN | TN | TN | 0 | 3 | 0 | 0 | 100% |
| ENTC | TN | N/A | N/A | 0 | 1 | 0 | 0 | 100% |
| NTC | TN | N/A | N/A | 0 | 1 | 0 | 0 | 100% |

The miRNA expression assay for prostate cancer demonstrated strong clinical performance across both inter-run and intra-run evaluations, confirming its reliability and diagnostic utility. A total of 264 individual miRNA calls were analyzed to determine the assay's sensitivity, specificity, and overall accuracy in distinguishing between high and low miRNA expression levels associated with disease status.

Sensitivity, defined as the assay's ability to correctly identify true positives (high miRNA expression in prostate cancer cases), was calculated at 93.8%. Out of 198 relevant calls, 185 were correctly identified as true positives, while 13 were classified as false negatives. This high sensitivity indicates that the assay reliably detects elevated miRNA expression in prostate cancer samples, minimizing the likelihood of missed diagnoses.

Specificity, which measures the assay's ability to correctly identify true negatives (low miRNA expression in non-cancer samples), was found to be 98.4%. Among 66 relevant calls, only one false positive was recorded, while 65 were true negative. This suggests a very low rate of false positive results, underscoring the assay's precision in excluding non-cancer cases.

Overall accuracy—the proportion of correct classifications (true positives and true negatives) out of all miRNA calls—was calculated at 94.6%. With 250 out of 264 calls accurately classified, these findings support the assay's clinical reliability and make it a strong candidate for use in regulated diagnostic settings, such as CLIA-certified laboratories.

What is claimed:

1) A gender-neutral method for detecting urological cancers in a urine sample from a patient comprising:
a) providing an unaltered urine sample
b) extracting miRNA from the urine sample
c) detecting miRNA biomarker levels in the urine sample to determine whether the patient is at risk for a urological cancer based on the levels of the miRNA biomarker.

2) The method of claim 1, wherein in the patient is male, female or non-binary.

3) The method of claim 1, wherein the urine sample is self-collected.

4) The method of claim 1, wherein the miRNA biomarker comprises hsa-let-7b5p, has-miR-26b5p, has-miR-1455p, has-miR-4253p, has-miR-1955p, has-miR-203a3p, has-miR-30c5p, has-miR-30a3p, or combinations thereof.
